(19) United States
(12) Patent Application Publication
(10) Pub. No.: US 2023/0366886 A1
(43) Pub. Date: Nov. 16, 2023

(54) SURFACE MODIFIED PARTICLES

(71) Applicants: USTAV ORGANICKE CHEMIE A BIOCHEMIE AV CR, V.V.I., Praha 6 (CZ); UNIVERZITA PALACKEHO V OLOMOUCI, Olomouc (CZ)

(72) Inventors: Petr CIGLER, Praha 6 (CZ); Jitka NEBURKOVA, Praha 6 (CZ); Jiri SCHIMER, Praha 8 (CZ); Miroslava GURICOVA, Zliechov (SK); Marian HAJDUCH, Moravsky Beroun (CZ); Hana JAWOREK, Olomouc (CZ); Martin ONDRA, Dolni Lutyne (CZ); Agata KUBICKOVA, Olomouc (CZ)

(21) Appl. No.: 18/028,669

(22) PCT Filed: Sep. 29, 2021
(86) PCT No.: PCT/CZ2021/050103

§ 371 Date: Mar. 27, 2023

(30) Foreign Application Priority Data
Sep. 29, 2020 (CZ) PV 2020-535

(51) Int. Cl. G01N 33/58; B82Y 15/00

(52) U.S. Cl. CPC G01N 33/587; B82Y 15/00

(57) ABSTRACT

Surface modified particles have a core, an inner shell and an outer shell. The core is formed of silica or is hollow, the inner shell is formed by a layer of metal, and the outer shell is formed by a biocompatible polymer brush. The particles allow for direct optical detection of biomolecules such as nucleic acids, proteins, polysaccharides and glycoproteins in biological samples.

### FIELD OF ART

The invention relates to particles allowing for direct optical detection of macromolecules for the purposes of in vitro diagnosis of diseases.

### BACKGROUND ART

Diagnostic methods are currently the basis for successful treatment of virtually all diseases. Laboratory examination of body fluid or tissue samples is often performed by so-called in vitro diagnostic methods. The thus obtained results are used, among other things, to search for patients suffering from the diseases (screening, for example for diabetes, prostate diseases, tumours of the colon and rectum), to determine or refine the diagnosis, to determine the prognosis or for epidemiological studies. A biological marker, (bio)marker, can be examined at the metabolic, genomic (DNA and RNA) or protein level. The most commonly used techniques in this field include immunohistochemistry (1HC), in situ hybridization techniques (ISH), and enzyme-linked immunosorbent assays (ELISA).

The principle of IHC is to determine an increased expression of a protein at the tissue or cellular level. The technical details of the procedure vary in certain parameters, for example in different incubation times, types of antibodies used, their dilution, etc. The technique is most often performed on paraffinized tissues, which are first deparaffinized and rehydrated. Non-specific binding of immunoglobulins is then blocked in an inert protein blocking solution. After washing, the samples are incubated with a primary anti-antibody. After washing, a secondary antibody, conjugated to a suitable detection system, is usually applied. Visualization is performed, for example, with the peroxidase enzyme and diaminobenzidine to produce a brown insoluble dye that visualizes an area of the tissue giving a positive signal. The sample is fixed under a coverslip and evaluated under a light microscope. The problem of IHC detection is, for example, the background of endogenous enzymatic activity (e.g. peroxidase activity in macrophages) as well as lower signal resolution (diffusion of the substrate during the enzymatic reaction) compared to, for example, fluorescent labelling.

ISH techniques have high specificity, reproducibility and speed of determination (within 24 hours). They can be used to determine the number of individual oncogenes in the cell nucleus. The samples are transferred to aqueous buffer and then denatured in formamide buffer, washed with ethanol and incubated with a labelled detection probe. Majority of these methods use the interaction of a synthetically prepared section of DNA (DNA detection probe), which carries a chemical or fluorescent label that allows direct visualization of the section of DNA. In the case of fluorescence labelling (fluorescence in situ hybridization, FISH), the positivity of the signal in the cells is read directly under a fluorescence microscope. In the case of a chromogenic in situ hybridization (CISH) enzymatic detection system, the DNA/RNA probe is labelled with a suitably modified nucleoside (e.g. biotin, a group suitable for bioorthogonal covalent conjugation, etc.) and detection is typically performed by an enzymatic system in a similar design with similar disadvantages as in the case of IHC. The signal can be read using brightfield microscopy. Due to the availability and low cost of light microscopes, the CISH technique is widely used in clinical diagnostics. However, the advantage of availability is redeemed by blurring of the signal due to the limitations of the enzymatic detection system, lower sensitivity of the method compared to FISH and more demanding sample preparation caused by incubation with enzymes generating the dyes.

There are currently a number of commercially available modified FISH probes labelled with various fluorescent labels, but there is no label that allows direct detection by visible light extinction.

The extinction intensity, and thus the colour contrast in the visible region provided by a given compound, can be quantified using the extinction coefficient ε_{λ}, where λ denotes the wavelength of light at which the extinction is measured. The colour of organic and inorganic molecules is typically caused by the absorption of radiation in the visible region, associated with the excitation of binding electrons. Conventional dyes have low ε_{λ} values ranging from about 1.10⁴ to 2.10⁵ M⁻¹ cm⁻¹. At such low values of ε_{λ} and real hybridization and amplification stoichiometries applicable in IHC, ISH or ELISA, it is not possible to observe localized contrast by light field microscopy. For the possibility of direct dye labelling in IHC, ISH or ELISA, the ε_{λ} of the dye would have to be at least 5-6 orders of magnitude higher.

In nanosystems made of precious metals (Au, Ag, Pt), the mechanism of radiation extinction is different. When light radiation interacts with a metal nanoparticle, the free photons in the metal lattice begin to oscillate in groups with the same frequency as the applied light. This phenomenon is known as localized surface plasmon resonance, which consists of two main contributions: 1) scattering, where incident light is emitted with the same energy but omnidirectionally, 2) absorption of photons forming a characteristic absorption band in the UV-vis spectrum, whose energy is converted into heat. These two contributions can be summarized as the extinction which is observed as the overall optical manifestation of metallic plasmonic nanosystems.

From the point of view of ISH detection, it is important that the molar extinction coefficients for the basic types of plasmonic nanoparticles are 4-5 orders of magnitude higher than for molecular dyes (Jain P. K. et al., J. Phys. Chem. B 2006, 110, 7238-7248). However, neither gold nor silver nanoparticles still have sufficient extinction properties for direct use. For IHC/ISH, the so-called metallographic detection based on horseradish peroxidase, which is conjugated to a secondary antibody, has recently been developed (Powell R. D. et al., Hum. Pathol. 2007, 38, 1145-1159). When Ag⁺ is present, it is reduced to metallic silver nanoparticles, which have more intense absorption and higher colour stability than molecular dyes. Another metallographic technique is to enhance the extinction of very small gold nanoparticles (<2 nm), which are bound by conjugates with antibodies to the target structure (Tubbs R. et al., J. Mol. Histol. 2004, 35, 589-594). The amplification is carried out by means of a secondary reduction of gold or silver induced preferably on the surface of the particles. The disadvantages of these metallographic methods are, similarly to molecular dyes, long incubation times required for the sequential binding of antibodies, time-consuming in situ reduction of metals, and high background caused by non-specific reduction in biomolecules.

Metal nanoshells are particles with a non-metallic core coated with a metal layer. They represent some of the strongest light absorbing and scattering nanostructures known in nature. Their molar extinction coefficient ε_{λ} reaches values of up to about 10¹² M⁻¹ cm⁻¹ and is approximately 7-8 orders of magnitude higher in comparison with molecular dyes. Nanoshells have hitherto been used, for example, for the detection and quantification of analytes by surface-enhanced Raman scattering, for example of glucose or proteins (US6699724B1). Furthermore, they have been used, for example, to release molecules from their surface, the release being caused by the heating of nanoshells by absorption of light in the near infrared region (US2020164072A1). For similar applications, anchoring the nanoshells in a crosslinked polymer gel or stabilizing the surface of the nanoshells by reacting the metal surface with thiolated poly(ethylene glycol), which is bound to the surface by metal-sulfur bonds, is sufficient. This polymer binding approach leads to a “mushroom conformation” of the polymer chain, which provides only basic colloidal protection.

It is known that for highly selective applications of nanoparticles in the biological environment, it is necessary to cover the surface of nanoparticles with a so-called biocompatible “polymer brush” (C. Cruje and D. B. Chithrani, Rev. Nanosci. Nanotechnol. 2014, 3, 20-30). This type of surface exhibits a high-area polymer density arrangement and provides effective protection against opsonization, non-specific adsorption of biomolecules on the particle surface and prevention of adsorption of nanoparticles on biological structures such as cell membrane and cell organelles. However, despite some efforts, such surface coating was not yet designed for metal nanoshells.

### DISCLOSURE OF THE INVENTION

The present invention solves the problem of direct detection in in vitro diagnostics by light extinction using particles with a non-metallic core coated with a metal layer, known as nanoshells, which are, however, specifically surface-modified.

From the point of view of IHC, ISH, and ELISA detection systems, the extinction properties of nanoshells reach an area where localized extinction due to the mere binding of nanoparticles to the target cell structure can be directly observed by light field microscopy (i.e. under favourable CISH-like instrumentation conditions) or using a plate reader (i.e. under ELISA-like instrumentation conditions). Unlike CISH, however, it is not necessary to use an enzymatic detection system, the main disadvantages of which are signal blur, lower sensitivity, low temperature stability and the need for long-term cold storage, frequent endogenous activity in the examined tissues, leading to increased background and/or decreased detection specificity. Furthermore, unlike ELISA, it is not necessary to use an enzymatic detection system, the main disadvantages of which are low temperature stability, the need for long-term cold storage and long development of the signal.

The present invention provides particles with a polymeric surface modification which allows their colloidal stabilization in solutions with ionic strength (buffers, media, blood, biological fluids) and in particular the suppression of non-specific interactions with biomolecules and biological interfaces. Furthermore, the method of their production and the method of attachment of molecules needed for selective recognition and subsequent visualization of detected biomarkers directly in tissues and cells are described.

The invention relates to surface-modified particles comprising a core, an inner shell and an outer shell, wherein
the core is formed of silica or the core is hollow (e.g., the core is a hollow cavity); and the core has a diameter d₁ in the range of 20 nm to 1 µm as determined by transmission electron microscopy (TEM),
the inner shell consists of a layer of metal M, said layer having a thickness d₂ in the range of 2 to 60 nm as determined by TEM,
the outer shell has a thickness d₃ in the range of 2 to 200 nm as determined by dynamic light scattering (DLS), and the outer shell consists of a layer of a polymer of general formula II

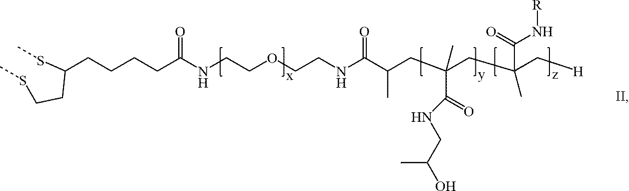

wherein x = 2 to 50, y = 5 to 5000, z = 0 to 2000, z/y = 0 to 0.4;
R are the same or different on each occurence, wherein each R is independently selected from the group consisting of —(CH₂)ₙ—C≡CH, —(CH₂)ₘ—N₃, —(CH₂)ₙ—NH₂, —(CH₂)ₙ—COOH,

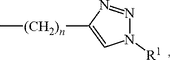

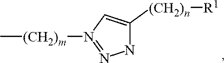

—(CH₂)ₘ—NH—C(O)—R², and —(CH₂)ₙ₋C(O)—NH—R¹,
wherein n = 1 to 4, m = 2 to 5;
R¹ is selected from the group consisting of

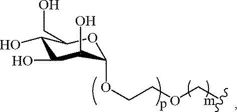

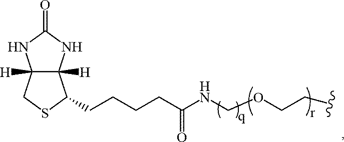

and a fluorophore,
wherein p = 0 to 24, q = 2 or 3, r = 0 to 24;
R² is selected from the group consisting of

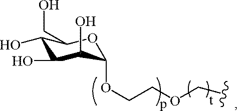

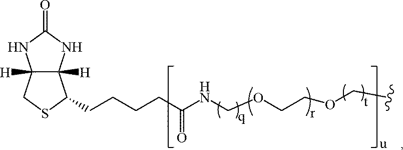

and a fluorophore,
wherein p = 0 to 24, q = 2 or 3, r = 0 to 24, t = 1 to 4, u = 0 or 1;
wherein the polymer of the general formula II is attached to the surface of the inner shell by means of its sulphur atoms forming an M-S bond with the metal atoms of the inner shell, as depicted by the dashed bonds in the formula II.

In some embodiments, preferably n = 1 to 2, and/or preferably m = 2 to 3, and/or preferably p = 0 to 12, and/or preferably r = 2 to 12, and/or preferably t = 1 to 3.

In some preferred embodiments, x = 3.

In the substituent R, the moiety —(CH₂)ₙ—C≡CH can be modified with an azide-containing compound R¹-N₃ to form

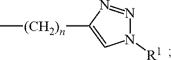

or the moiety —(CH₂)ₘ—N₃ can be modified with an alkyne-containing compound R¹—(CH₂)ₙ—C≡CH to form

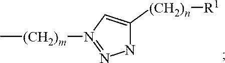

or the moiety —(CH₂)ₘ₋NH₂ can be modified with a carboxylic acid R²—COOH to form —(CH₂)ₘ₋NH—C(O)—R²; or the moiety —(CH₂)ₙ—COOH can be modified with an amine R¹—NH₂, to form —(CH₂)ₙ—C(O)—NH—R¹.

When R¹ is

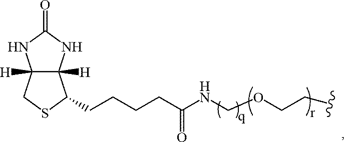

the biotin moiety (biotin residue) may optionally be conjugated to a biotin-binding protein such as neutravidin, streptavidin, or avidin, via formation of a non-covalent attachment between biotin and the biotin-binding protein (i.e., via formation of a non-covalent complex of the biotin moiety and the biotin-binding protein).

When R¹ is

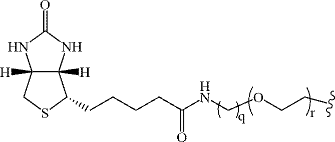

and it is conjugated to the biotin-binding protein, a biotin-modified biomolecule may optionally be further non-covalently attached to the biotin-binding protein via formation of a non-covalent attachment between the biotin-binding protein and the biotin moiety of the biotin-modified biomolecule (i.e., via formation of a non-covalent complex of the biotin moiety and the biotin-binding protein). The biotin-modified biomolecule may be, for example, a biotin-modified protein, more specifically a biotin-modified antibody.

Preferably, the metal M forming the inner shell is selected from the group consisting of gold, silver, nickel and copper.

Particularly preferably, the metal M forming the inner shell is gold (Au).

The inner shell preferably has a thickness d₂ in the range of 3 to 25 nm, more preferably 5 to 20 nm.

The outer shell preferably has a thickness d₃ in the range of 5 to 100 nm, more preferably 10 to 60 nm.

The polymer of formula II forming the outer shell serves to suppress non-specific interactions of modified particles in the biological environment (ie. as a so-called polymer brush) and to attach molecules enabling selective recognition and subsequent visualization of detected biomarkers directly in tissues, cells and biological samples.

In the particles of the present invention, the core, the inner shell and the outer shell are usually arranged substantially concentrically. They may preferably be substantially spherical.

The invention further relates to a process for the preparation of surface-modified particles, in which the particles containing the core and the inner shell are reacted with a compound of formula III

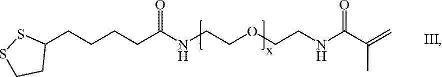

wherein x is as defined above,
wherein the compound of formula III is optionally in a mixture with lipoic acid in a molar ratio of lipoic acid: compound of formula III = 2:1 to 6:1, preferably 4:1, and and the product is subsequently contacted with monomer of formula IV under free radical polymerization conditions

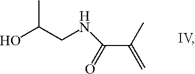

wherein the monomer of formula IV optionally contains an admixture of 0 to 40 molar % of monomer of formula V

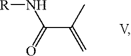

wherein R is as defined above, to form a polymer of formula II

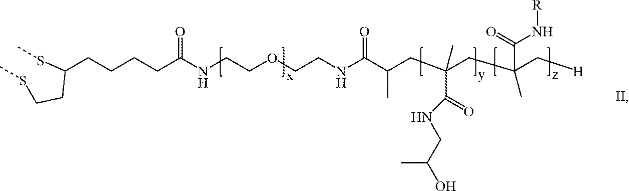

wherein R, x, y, z are as defined above,
attached to the inner shell of the particles, thereby forming the outer shell.

The invention also relates to an alternative process for the preparation of surface-modified particles, in which a compound of formula IIA

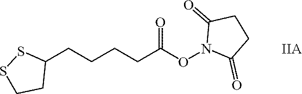 [SMILES: O=C(CCCCC1CCSS1)ON1C(=O)CCC1=O]

is reacted with polymer of formula IIB

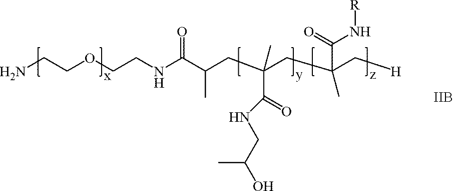

to form polymer of formula IIC

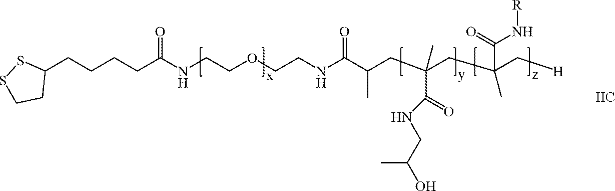

wherein R, x, y, z are as defined above, which binds in an aqueous medium to particles containing a core and the inner shell, to form particles with bound polymer II forming the outer shell.

The particles containing silica core and gold inner shell can be preferably prepared as follows:
in a first step, silica cores are modified by reaction with trialkoxysilane derivatives of formula R⁴Si(R³)₃, wherein R⁴ is selected from C₂-C₄ alkyl terminally substituted with mercapto or amino group, and R³ are selected from the group consisting of —OCH₃ and —OCH₂CH₃,
in a second step, gold nanoparticles with a diameter of less than 5 nm are bound to the thus modified core particles,
in a third step, the resulting particles, formed by a core with bound gold nanoparticles, are reacted with [AuCl₄⁻] in the presence of a reducing agent, thus forming an inner shell.

Preferably, in the first step, the trialkoxysilane derivatives are selected from the group consisting of (3-mercaptopropyl)trimethoxysilane, (3-mercaptopropyl)triethoxysilane, (3-aminopropyl)-trimethoxysilane, and (3-aminopropyl)triethoxy silane.

Most preferably, (3-mercaptopropyl)trimethoxysilane is used in the first step.

Preferably, in the third step, the reducing agent is selected from the group consisting of carbon monoxide, hydroxylamine, hydrazine, methylhydrazine, ascorbic acid, formaldehyde and acetaldehyde.

In some embodiments, the particles containing hollow core and gold inner shell can be preferably prepared as follows:
in a first step, (3-aminopropyl)triethoxysilane or (3-aminopropyl)trimethoxysilane is stirred with water,
in a second step, HAuCl₄ is added followed by addition of NaBH₄, to form particles, preferably HAuCl₄ and NaBH₄ are added in the form of solution(s),
in a third step, the formed particles are stabilized by bovine serum albumine solution.

If compounds II to V contain chiral centres, then formulas II to V include pure enantiomers as well as mixtures of enantiomers, including racemates.

The presence of reactive azide, alkyne, amine or carboxylic acid groups in the R substituent of polymer II allows easy attachment of other molecules and/or biomolecules to surface-modified particles, which can then be used to selectively recognize and visualize detected biomarkers directly in tissues, cells and biological samples. The molecules can also further improve the properties of the particles, for example, fluorophores can introduce the ability of the particles to fluoresce.

In a preferred embodiment, the derivatization or the formation of the substituent R¹ is carried out by reacting the reactive group of the azide, alkyne, amine or carboxylic acid in the substituent R in polymer II with its reaction complement, i.e. for example azide with alkyne to form a triazole, alkyne with azide to form a triazole, amine with a carboxylic acid to form an amide, a carboxylic acid and an amine to form an amide.

In particular, surface-modified particles can be derivatized for conjugation to recognition structures by the biotin-streptavidin, biotin-avidin or biotin-neutravidin system.

This derivatization is carried out by reacting the reactive alkyne group in polymer II with an azided biotin derivative, e.g. biotin-PEG₁₁-azide, catalyzed by copper ions. This results in surface-modified particles bearing biotin, which can bind to a target site containing a biotin-binding protein such as avidin, streptavidin or neutravidin. Biotin-modified particles can be further derivatized with an excess of biotin-binding protein such as avidin, streptavidin, or neutravidin to provide a biotin-binding surface. Biomolecules modified by biotin can be then attached to the particles upon formation of a non-covalent complex with the biotin-binding protein.

In a preferred embodiment, the biomolecules modified by biotin (i.e. biotinylated biomolecules) can be proteins and nucleic acids or a part thereof.

Biotinylated proteins are routinely available on the market. For example, biotinylated proteins can be commercially available biotinylated antibodies such as immunoglobulins, for example biotinylated immunoglobulins A, G, D, E, and M.

The term “nucleic acid or a part thereof” is understood to mean nucleic acids or their segments selected preferably from oligonucleotides, deoxyribonucleic acid (single-stranded DNA, double-stranded DNA), and ribonucleic acid. Furthermore, all nucleic acids disclosed herein may be formed or modified with synthetic base analogs, for example, to increase their stability. Synthetic analogs of nucleic acids involve, in particular, the following substitutions: phosphorylation at the 5′ and/or 3′ end of the strand, 5-methylcytidine-5′-triphosphate, N¹-methylpseudouridine-5′-triphosphate, P¹-(5′-(3′-O-methyl)-7-methyl-guanosyl)-P³-(5′-(guanosyl))triphosphate, P¹-(guanosyl) P³-(5′-(guanosyl))tri-phosphate, P¹-(5′-7-methyl-guanosyl) P³-(5′-(guanosyl))triphosphate, P¹-(5′-2,2,7-trimethyl-guanosyl) P³-(5′-(guanosyl))triphosphate, N⁶-methyladenosine-5′-triphosphate, 2-thiouridine-5′-triphosphate, pseudouridine-5′-triphosphate, 5-methoxyuridine-5′-triphosphate, N¹-methyladenosine-5′-triphosphate, N⁴-acetylcytidine-5′-triphosphate, 2′-O-methyl, 2′-O-methoxyethyl, 2′-fluoro, a methylene bridge between the 2′-oxygen and the 4′-carbon of the pentose ring (a so-called locked nucleic acid), boranophosphonates, or phosphorothioates.

Fluorophores are molecules providing fluorescence. The structure of fluorophores can be proprietary. The fluorophores are commercially available in form of derivatives containing already the reactive groups, sometimes connected to the fluorophore via molecular linkers, which can be used for attachment to the particles, more particularly to their azide, alkyne, amine or carboxylic acid groups. The carboxylic acid group can be used for attaching a fluorophore either as a free acid or in form of an active ester such as N-hydroxysuccinimidyl ester, sulfo-N-hydroxysuccinimidyl ester or pentafluorophenyl ester. Non-limiting examples of fluorophores which can be attached to the particles, include Alexa Fluor 350, Alexa Fluor 405, Alexa Fluor 488, Alexa Fluor 532, Alexa Fluor 546, Alexa Fluor 555, Alexa Fluor 561, Alexa Fluor 568,, Alexa Fluor 594, Alexa Fluor 647, Alexa Fluor 660, Alexa Fluor 680, Alexa Fluor 700, Alexa Fluor 750, AMCA, BODIPY® 630/650, BODIPY® 650/665, BODIPY®-FL, BODIPY®-R6G, BODIPY®-TMR, BODIPY®-TRX, Cascade Blue®, CyDye™, including but not limited to Cy2™, Cy3™, and Cy5™, 6-FAM™, Fluorescein, HEX™, 6-JOE, Oregon Green® 488, Oregon Green® 500, Oregon Green® 514, Pacific Blue™, REG, Rhodamine Green™, Rhodamine Red™, ROX™, TAMRA™, TET™, Tetramethylrhodamine, Texas Red®, Pacific Green, Pacific Orange, PE-Cyanine7, PerCP-Cyanine5.5, eFluor 450, eFluor 506, eFluor 660, PE-eFluor 610, PerCP-eFluor 710, APC-eFluor 780, Super Bright 436, Super Bright 600, Super Bright 645, Super Bright 702, Super Bright 780, Qdot 525, Qdot 565, Qdot 605, Qdot 655, Qdot 705, Qdot 800, Allophycocyanin (APC), R-Phycoerythrin (R-PE), Cyan Fluorescent Protein (CFP), Green Fluorescent Protein (GFP), Red Fluorescent Protein (RFP), Brilliant Ultra Violet 737, Brilliant Ultra Violet 805, NovaFluor Blue 510 Dye, NovaFluor Blue 530 Dye, NovaFluor Blue 555 Dye, NovaFluor Blue 610-30S Dye, NovaFluor Blue 610-70S Dye, NovaFluor Blue 660-40S Dye, NovaFluor Blue 660-120S Dye, NovaFluor Yellow 570 Dye, NovaFluor Yellow 610 Dye, NovaFluor Yellow 660 Dye, NovaFluor Yellow 690 Dye, NovaFluor Yellow 700 Dye, NovaFluor Red 660 Dye, NovaFluor Red 685 Dye, or NovaFluor Red 700 Dye.

In another preferred embodiment, the reactive alkyne group in polymer II is reacted with an azided mannose derivative, e.g. mannose-PEG₄-azide, catalyzed by copper ions. This results in surface-modified particles bearing mannose that can bind to a target site containing a mannose-recognizing receptor, e.g. CD206, highly expressed on the macrophage membrane. After incubation of such modified nanoparticles with macrophages, it is possible to analyze the phagocytic activity of macrophages by flow cytometry only by means of increased light scattering caused by the modified nanoparticles, without the use of fluorescent labels.

By varying the geometric parameters of surface-modified particles (core diameter, inner shell thickness, total particle diameter), different particles with different optical properties can be prepared (molar extinction coefficient, plasmonic extinction band position, ability to create contrast in transmitted light microscopy). The starting core particles can be prepared by reactions and procedures known to those skilled in the art, and some silica particles are also commercially available.

Surface-modified particles are useful for a variety of in vitro diagnostic applications. These applications involve the detection of biomolecules in biological samples based on the interaction of modified particles with these biomolecules. The biomolecule may be selected from the group consisting of nucleic acids, proteins, polysaccharides and glycoproteins. By biological sample is meant, for example, blood, blood plasma, blood serum, urine, semen, tears, saliva, mucus, stool, sweat, swab, lymph, cerebrospinal fluid, cell suspension, tissue.

In a preferred embodiment, the surface-modified particles can also be used in a system for detecting an integrated form of HPV16 virus in the genome of human cervical cancer cells. In this embodiment, the digoxigenin-labeled gene probe is hybridized to cell DNA and subsequently modified with a biotinylated anti-rabbit immunoglobulin antibody. The presence of viral DNA is then visualized using surface-modified neutravidin-derivatized nanoparticles and manifests itself as dark contrast spots on a light background. The detection performed in this way enables fast, accurate and very sharp identification of the presence of HPV16 in tumour cells.

To visualize proteins, it is possible to derivatize surface-modified nanoparticles using ligands that bind to these proteins.

Thus, the present invention provides particles with a non-metallic core coated with a metal layer and a surface that is modified with a biocompatible polymer brush. This solution provides advantages in imaging target structures in tissues in a conventional light microscope, namely direct and easy detection, a sharper, less diffuse signal and chemical and biological long-term stability of nanoparticles with a wide range of applications in diagnostics and bioanalysis. The present invention further provides advantages in the field of flow cytometry and haemocytometry, where the extreme scattering caused by the particles after their uptake into the cell allows direct identification and quantification of such labelled cells only by light scattering, without the need for fluorescence detection. The optical properties of the particles - the position and width of the plasmonic absorption band - can be tuned for each application by the size and geometry of the particles, the composition and size of the non-metallic core and the thickness of the shell.

### BRIEF DESCRIPTION OF DRAWINGS

FIGS. 1A-1C: Extinction spectra of modified particles FIG. 1A) A6, A7 and A8, FIG. 1B) B6 and B7, and FIG. 1C) C4, normalized to the absorption maximum. Individual surface modifications do not affect the position of the spectral maxima of series A and series B.

FIGS. 2A-2B: Dynamic light scattering for modified particles FIG. 2A) A6, A7 and A8, FIG. 2B) B5, B6 and B7 measured in water, PBS buffer (PBS) and 10x concentrated PBS buffer (10x PBS). For both series of particles A and B, a high colloidal stability is evident, characterized by a stable position of the distribution histogram, which is independent of the environment. Exposure of the modified particles to 10x PBS is a robust assay that corresponds to an ionic strength approximately an order of magnitude higher than that present in a physiological environment.

FIG. 3: Micrographs of modified particles A1, A3, A4, B1, B3, B4 and C4 from a transmission electron microscope. For each of the series A and B, the gradual growth of the particle is demonstrated: A1 and B1 are the starting silica particles, A3 and B3 are silica particles with bound gold nanoparticles, A4 and B4 are compact modified particles. Scale sizes: for A1, A3, A4 particles, the scale is always 100 nm. For particle B1 200 nm, for B3 100 nm, for B4 and C4 200 nm.

FIG. 4: Detection of human papillomavirus (HPV16) in cervical carcinoma (SiHa) cells by in situ hybridization of viral DNA with detection by modified particles A8. The specific HPV16 signal is visualized by light field microscopy as dark, high contrast spots on a light background located in the nucleus region. In the practical embodiment, specific colors are used: the nucleus is colored with nuclear red (pink color of the nucleus), visualization with A8 provides a dark blue color. Magnification 20x.

FIGS. 5A and 5B: Analysis of phagocytosis by J774A.1 murine macrophages by flow cytometry without fluorescent labeling. FIG. 5A) Distribution of cells after 3 hours in a control group without modified particles (Control), with modified particles (B6) and with modified particles derivatized with mannose (B7). The process of phagocytosis was monitored using,,dot plots” visualizing the size (direct light scattering, FSC-A) and granularity (lateral light scattering, SSC-A) of individual cells. FIG. 5B) Dynamics of cell distribution in individual quadrants. The specific uptake of modified particles by macrophages is manifested by a significant increase of the signal in quadrant Q1, which also increases with time.

FIG. 6: Light field microscopy of live macrophages J774A.1 for times 0 to 12 hours. The following variants were tested: medium without particles (Control), medium containing control modified particles B6 carrying only polymer (negative control) and modified particles B7 derivatized with mannose (both particles at a final concentration of 7.3 pmol.1⁻¹). Phagocytosis was clearly observable only for the specific variant containing B7 with bound mannose as a significant change in optical contrast in the cell areas.

FIG. 7: Light field microscopy of PBMC cells were isolated from whole blood after 48 h incubation at 4° C. The following variants were tested: medium without particles (Control), medium containing control modified particles C8 carrying only polymer (negative control), modified particles C9 derivatized with CD3 biotinylated monoclonal antibody, and modified particles C10 derivatized with CD4 biotinylated monoclonal antibody. The individual cells containing the corresponding receptors (either CD3 or CD4) show a significant change in optical contrast in the cell areas manifested as dark spots. Notably, not all cells are stained, because PBMC cells contain only a certain fraction of either CD3 and/or CD4 positive cells.

FIG. 8 schematically shows the structure of a surface-modified particle comprising a core 1. an inner shell 2 and an outer shell 3, where the core 1 is made of silica or it is hollow and has a diameter dₜ, the inner shell 2 is formed by a metal layer M of thickness d₂, and the outer shell 3 of thickness d₃ is formed by a layer of polymer.

List of Abbreviations

| | |
|---|---|
| DCM | dichloromethane |
| DIPEA | N,N-diisopropylethylamine |
| UPLC-MS | ultra high performance liquid chromatography-mass spectrometry |
| TLC | thin layer chromatography |
| MeOH | methanol |
| DMSO | dimethylsulfoxide |
| HRMS | high resolution mass spectrometry |
| UPLC | ultra-high performance liquid chromatography |
| QDa | quadrupole detector in mass spectrometry |
| ACN | acetonitrile |
| PEG | polyethyleneglycol |
| BTTAA | 2-(4-((bis((1-(tert-butyl)-1H-1,2,3-triazol-4-yl)methyl)amino)methyl)-1H-1,2,3-triazol-1-yl) acetic acid |
| V-601 | dimethyl 2,2′-azobis(2-methylpropionate) |
| PBMC | peripheral blood mononuclear cells |

### EXAMPLES

### Example 1

### Synthesis of Ligand for Polymerization

500 mg of Boc-PEG₄-NH₂ (1.0 eq.) was dissolved in 50 ml of DCM and 446 µl (1.5 eq.) of DIPEA was added to the reaction mixture, which was subsequently cooled in an ice bath. 167 µl (1.0 eq.) of methacryloyl chloride was added dropwise over about 5 minutes. The reaction was allowed to gradually warm to room temperature and stirred overnight. In the morning, the reaction mixture was shaken twice with 10% (always volume percentage, unless otherwise stated) KHSO₄, twice with saturated sodium bicarbonate solution and once with brine, 30 ml each time. The organic phase was then dried over anhydrous magnesium sulphate and evaporated on an evaporator. According to UPLC-MS analysis of the crude product, the residue had a purity of more than 95 % (Rt = 3.7 min, identified m/z: 361.4 [M+H]⁺, 261.3 [M-Boc, +H]⁺. TLC analysis (pure EtOAc, R_{f} = 0.3) confirmed, after staining with 3% KMnO₄, the only major substance present. Product VI was used in the next step without any further purification.

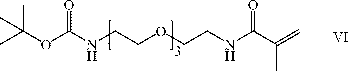

The crude evaporated product VI was dissolved in 2 ml of trifluoroacetic acid (TFA) and the solution was sonicated in a bath for 5 min to promote deprotection of the Boc protecting group. The TFA was then evaporated in a stream of nitrogen. After complete removal of TFA, the resulting product VII was used in the next step without further purification.

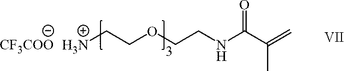

639 mg (1.0 eq) of compound VII was dissolved in 50 ml of DCM and then 1.06 ml (3.5 eq) of DIPEA was added. The pH of the reaction mixture was checked to be basic (if the TFA was not removed sufficiently, more DIPEA was needed). 528 mg (1.0 eq) of N-hydroxysuccinimidyl lipoic acid ester was added in one portion and the reaction was stirred overnight. Chromatography on preparative TLC plates (Analtech P02015 Silica GF UNIPLATE, 2000 µm, 200 mm wide, 200 mm length, 4 plates used for separation) was performed as the only purification method (5% MeOH in DCM, R_{f} = 0.5, UV active). The spot containing the product was scraped off and the product was extracted on a frit with pure MeOH. The substance was further used in the form of a solution in MeOH, because evaporation and concentration of the solution led to spontaneous polymerization. Only a small portion of the solution was evaporated to determine the concentration. 390 mg of product VIII was isolated in MeOH solution (final isolated yield after 3 steps was 51 %). This solution was stable and stored for many months in a freezer at -80° C. UPLC-MS analysis detected only one peak (R₁ = 3.7 min, purity 97 %).

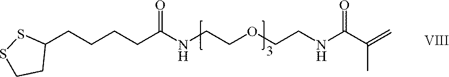

For NMR analysis, the solution was gradually evaporated into DMSO. ¹H NMR (400 MHz, DMSO-d₆) δ 7.92 (NH, t, J = 5.7 Hz, 1H), 7.84 (NH, t, J = 5.6 Hz, 1H), 5.65 (CH₂=, dq, J = 1.9, 1.0 Hz, 1H), 5.32 (CH₂=, h, J = 1.5 Hz, 1H), 4.14 (q, J = 5.2 Hz, 3H), 3.66 - 3.56 (m, 1H), 3.54 - 3.47 (m, 4H), 3.46-3.39 (m, 1H) 3.26 (q, J = 6.1 Hz, 2H), 3.21 - 3.15 (m, 6H), 3.14-3.06 (m, 2H), 2.41 (dtd, J = 12.9, 6.5, 5.5 Hz, 1H), 2.07 (t, J = 7.3 Hz, 2H), 1.93 - 1.79 (m, 4H), 1.66 (dtd, J = 13.6, 7.9, 7.4, 5.5 Hz, 1H), 1.58 - 1.42 (m, 3H), 1.40 - 1.28 (m, 2H). ¹³C NMR (101 MHz, DMSO-d₆) δ 172.63, 168.06, 140.30, 119.57, 70.18, 70.03, 69.60, 69.26, 56.62, 38.92, 38.55, 35.57, 34.56, 28.74, 25.48, 19.06. HRMS calculated for C₂₀H₃₇O₅N₂S₂: m/z [M+H]⁺: 449.21384; found 449.21378.

UPLC-MS analysis: data were obtained on a Waters acquity UPLC system connected to a qDA detector. A 2.1×100 mm C18 1.7 µm Acquity Waters was used as the column. The 7-minute gradient was set as follows: 2% ACN for 1 minute, then gradient up to 100% ACN over the next 5 minutes, followed by 1 minute of pure ACN.

### Example 2

### Preparation of Modified Particles With Silica Core

### A) Preparation of Silica Particles

Silica particles were prepared in two steps. First, particle seed grains were prepared. 4.55 mg of arginine was dissolved in 3.45 ml of water. The solution was overlaid with a mixture of cyclohexane (225 µl) and tetraethoxyorthosilicate (275 µl). The mixture was heated to 60° C. and stirred for 20 hours. Then, the lower phase containing the silica seed grains was separated.

For further growth of silica particles of various sizes, 39.5 ml of ethanol, 11 ml of H₂O and 466 µl of the prepared solution of silica seed grains were mixed. After thorough mixing, tetraethoxyorthosilicate and 0.9 ml of 24.5% NH₄OH solution were added with stirring. The amount of tetraethoxyorthosilicate was varied according to the desired particle size. For example, to prepare A1 particles with an average core diameter d₁= 58 nm, 180 µl was added; 1000 µl was added to prepare larger B1 particles with an average core diameter d₁ = 95 nm. The reaction was run overnight at room temperature. The particles were washed with ethanol.

### B) Preparation of Thiolated Silica Particles

Particles A1 or B1 (10 mg) prepared in the previous step were mixed with 11.5 ml of water, 37.5 ml of ethanol and the solution was purged with argon for 10 minutes. With stirring, solutions of (3-mercaptopropyl)trimethoxysilane and 1,2-bis(triethoxysilyl)ethane were added in amounts suitable for the given silica particle sizes (to form A2: 14.5 µl to A1 particles; to form B2: 8 µl to particles B1). While stirring, 0.9 ml of 24.5% NH₄OH solution was added and the reaction mixture was allowed to react for 4 hours at room temperature under an inert atmosphere of argon. The prepared thiolated particles were washed with ethanol.

### C) Preparation of Silica Particles With Bound Gold Nanoparticles

First, gold nanoparticles with a diameter of <5 nm were prepared by adding 2 ml of a 1% solution of terachloroauric acid to a solution of tetrakis(hydroxymethyl)phosphonium chloride (1.34 µmol.1⁻¹ in 50 ml of 10 mmol.1⁻¹ NaOH solution). The thiol-modified particles (A2, B2) were mixed with this solution. The resulting particles with bound gold nanoparticles (A3, B3) were washed with water.

### D) Growth of the Inner Shell

Particles A3 or B3 were mixed with a gilding solution (250 mg K₂CO₃, 174 mg HAuCl₄, 1 litre water, pH 9.5) and bubbled with carbon monoxide. The ratio of the amount of particles and the gilding solution depended on the particle size and the required thickness of the gold layer. For 0.5 mg of A3 particles, 72 ml of solution was used to form A4 particles, for 0.5 mg of B3 particles, 40 ml of gilding solution was used to form B4. The particles were separated by sedimentation. The average diameter of the inner shell was d₂ = 14 nm for A4 particles and d₂ = 17.5 nm for B4 particles.

### Example 3

### Preparation of Modified Particles With Hollow Core

The gold nanoshells with hollow core were prepared according to modified procedure of Guan, Y. et al. (Colloid Surface A, 2016, 502, 6-12). (3-aminopropyl)triethoxysilane (2.0 ml) was mixed with milliQ water (920 ml) under constant stirring. After 10 s, a solution of HAuCl₄ (20 ml, 25 mmol.1⁻¹) was added to the mixture to form yellow suspension. The reaction mixture was stirred for 30 s, followed by addition of freshly prepared NaBH₄ solution (80 ml, 0.1 mol.1⁻¹). After addition of NaBH₄, the suspension changed color from yellow to blue. The formed particles with hollow core of average diameter d₁ = 68 nm and diameter of the inner shell d₂ = 12 nm were stabilized by addition of solution of bovine serum albumine (80 ml, 0.1 mmol.1⁻¹).

### Example 4

### Preparation of Particles With a Layer of Hydrophilic Polymer

A mixture of ligand VIII and lipoic acid (14.4 ml of 10 mmol.1⁻¹ lipoic acid and 3.6 ml of 10 mmol.l⁻¹ ligand VIII) was added to the gold-plated particles A4 diluted in 700 ml of water, the pH was adjusted to 9.5. Ligand exchange lasted 3 days. The resulting A5 particles were concentrated by sedimentation.

A mixture of ligand VIII and lipoic acid (9.64 ml of 10 mmol.1⁻¹ lipoic acid and 2.41 ml of 10 mmol.1⁻¹ ligand) was added to the B4 particles diluted to 500 ml, the pH was adjusted to 9.5. Ligand exchange lasted 3 days. The resulting B5 particles were concentrated by sedimentation.

A mixture of ligand VIII and lipoic acid (9.64 ml of 10 mmol.1⁻¹ lipoic acid and 2.41 ml of 10 mmol.1⁻¹ ligand) was added to the C4 particles diluted to 500 ml, the pH was adjusted to 9.5. Ligand exchange lasted 3 days. The resulting C5 particles were concentrated by sedimentation.

A concentrated solution of A5 particles (45 ml corresponding to 5 mg of silica particles) was mixed with a solution of HPMA (9.72 g in 84 ml of water, pH 11) with constant stirring and purged with argon. A mixture of monomer and initiator in methanol was added to the solution under argon [486 µl alkyl of alkyne monomer N-(prop-2-yn-1-yl)methacrylamide, 466 µl of initiator V-601 (Fujifilm, cat. Nr. LB-V601-20GS) in 14.6 ml of methanol]. The mixture was heated to 60° C. for 48 hours. The resulting A6 particles were washed with methanol and then PBS with 0.1% (w/w) Tween detergent.

A concentrated solution of B5 particles (30 ml corresponding to 6.75 mg of silica particles) was mixed with a solution of HPMA (5 g in 53.75 ml of water, pH 11) with constant stirring and purged with argon. A mixture of monomer and initiator in methanol [250 µl of N-(prop-2-yn-1-yl)methacrylamide, 300 µl of initiator V-601 in 9.375 ml of methanol] was added to the solution under an inert atmosphere of argon. The mixture was heated to 60° C. for 48 hours. The resulting B6 particles were washed with methanol and then PBS with 0.1% (w/w) Tween detergent.

A concentrated solution of C5 particles (30 ml corresponding to 6.75 mg of silica particles) was mixed with a solution of HPMA (5 g in 53.75 ml of water, pH 11) with constant stirring and purged with argon. A mixture of monomer and initiator in methanol [250 µl of N-(prop-2-yn-1-yl)methacrylamide, 300 µl of initiator V-601 in 9.375 ml of methanol] was added to the solution under an inert atmosphere of argon. The mixture was heated to 60° C. for 48 hours. The resulting C6 particles were washed with methanol and then PBS with 0.1% (w/w) Tween detergent.

The thickness of the polymer outer shell for the particles A6, B6, and C6 was approximately d₃ = 15 nm.

### Example 5

### Derivatization of Modified Particles Using Biotin and Neutravidin

A6 particles weighing 180 µg of silica core were modified with biotin by copper ion catalyzed azide-alkyne cycloaddition. The particles were diluted to 600 µl and mixed with 1 µl 12.5 mmol.1⁻¹ biotin-PEG₁₁-azide (BroadPharm, USA, cat. Nr. BP-21626, CAS 956494-20-5). In a final volume of 700 µl, reagent concentrations were used: 77 µmol.1⁻¹ CuSO₄, 155 µmol.1⁻¹ BTTAA ligand (Sigma-Aldrich, cat Nr. 906328, CAS 1334179-85-9), 3.86 mmol.1⁻¹ sodium ascorbate and 3.86 mmol.l⁻¹ aminoguanidine. The reaction was allowed to react for 2 h and then the A7 particles were washed 7 times in PBS. Half of the A7 particles were mixed with 200 µl of neutravidin in PBS (1 mg/ml) and allowed to react at 4° C. overnight. Particles A8 were washed in PBS with 0.1% (w/w) Tween detergent.

### Example 6

### Derivatization of Modified Particles Using Mannose

B6 particles were modified with mannose by copper ion catalyzed azide-alkyne cycloaddition. The particles were diluted to 4.5 ml and mixed with 377 µl of 10 mmol.1⁻¹ mannose-PEG₄-azide (BroadPharm, USA, cat. Nr. BP-23832, CAS 1632372-86-1) (0.74 mmol.1⁻¹). In the final volume of 5089 µl, there was 0.15 mmol.1⁻¹ CuSO₄, 0.3 mmol.1⁻¹ BTTAA ligand, 1.48 mmol.1⁻¹ ascorbate and 1.48 mmol.1⁻¹ aminoguanidine. The reaction was allowed to react for 2 hours and then the mannosylated B7 particles were washed 7 times with PBS with 0.1% (w/w) Tween detergent.

### Example 7

### Derivatization of Modified Particles Using Antibodies

C6 particles were modified with biotin by copper ion catalyzed azide-alkyne cycloaddition. The particles were diluted to 600 µl and mixed with 1 µl 12.5 mmol.1⁻¹ biotin-PEG₁₁-azide. In a final volume of 700 µl, reagent concentrations were used: 77 µmol.1⁻¹ CuSO₄, 155 µmol.1⁻¹ BTTAA ligand, 3.86 mmol.1⁻¹ sodium ascorbate and 3.86 mmol.1⁻¹ aminoguanidine. The reaction was allowed to react for 2 hours and then the biotinylated C7 particles were washed 7 times in PBS. Half of the C7 particles were mixed with 200 µl of neutravidin in PBS (1 mg/ml) and allowed to react at 4° C. overnight. The resulting neutravidin-modified particles C8 were washed in PBS with 0.1% (w/w) Tween detergent.

Particles C8 were incubated with biotinylated immunoglobulin G - CD3 monoclonal antibody (OKT3) (ThermoFischer, cat. Nr. 13-0037-82) for 1 h at room temperature. Afterwards, the resulting particles were washed with 1x PBS with 0.1% Tween 20 and centrifuged at 1000 rcf for 10 min. The supernatant was removed and again centrifuged at 2000 rcf for 10 min, supernatant was discarded. The resuspended pellets were pooled together and the procedure was repeated three times providing particles C9 modified with CD3 monoclonal antibody.

Another portion of particles C8 was incubated with biotinylated immunoglobulin G - CD4 monoclonal antibody (OKT4) (ThermoFischer, cat. Nr. 13-0048-82) and purified at the same conditions as used above for preparation of C9. The procedure yielded particles C10 modified with CD4 monoclonal antibody.

### Example 8

### Characterization of Modified Particles

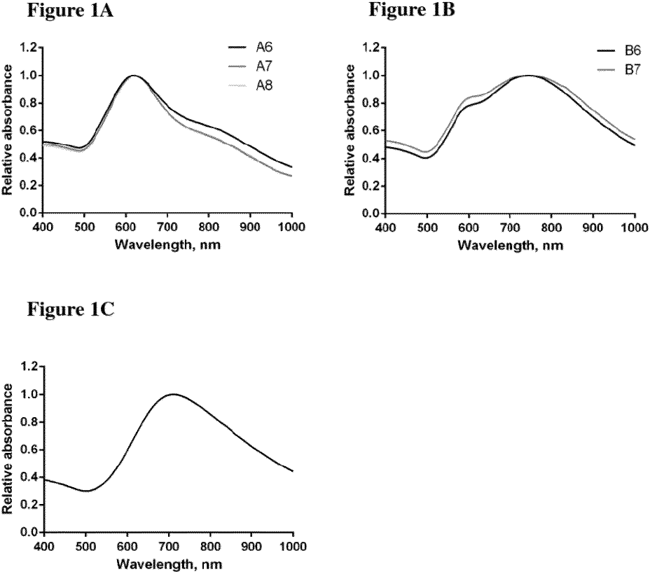

The extinction spectrum of the prepared modified particles was measured (FIG. 1). At the low picomolar concentration used, all particles showed intense absorption bands in the region suitable for signal detection.

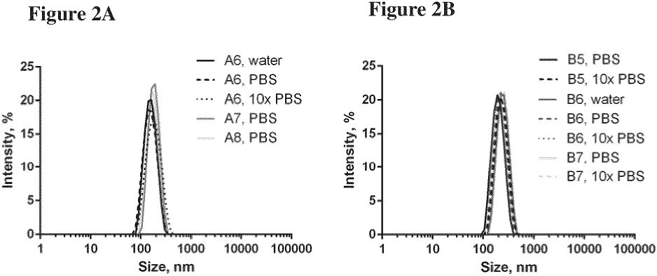

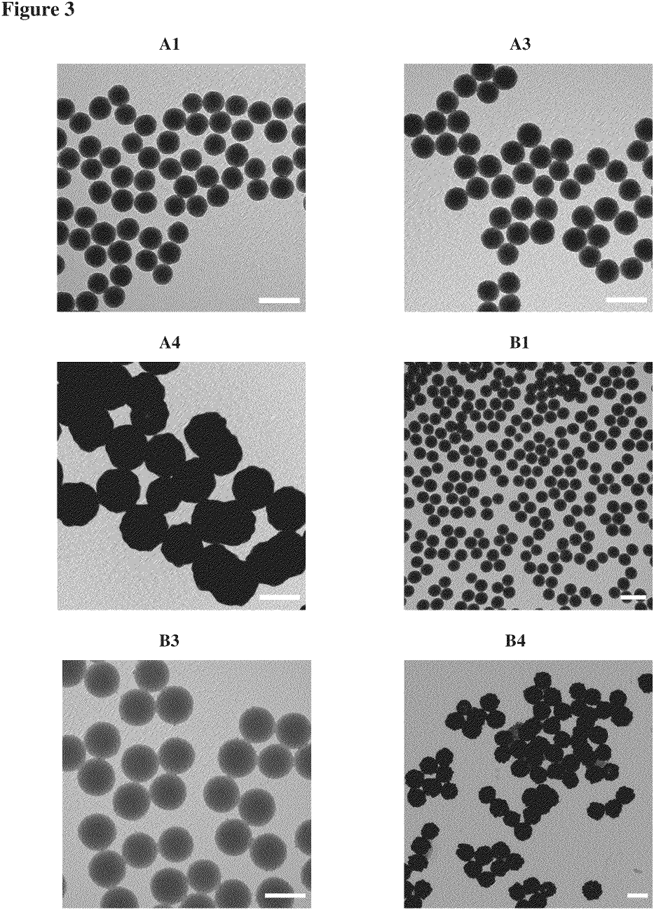

The dynamic light scattering of the prepared modified particles was measured at concentrations corresponding to units of pmol.l⁻¹ in water, in PBS buffer and 10x concentrated PBS buffer, which due to its high ionic strength represents a very robust test of colloidal stability (FIG. 2). All modified particles showed high stability even in the environment of 10x concentrated PBS buffer, which indicates a strong stabilizing function of the polymer protecting the interaction interface of the modified particles.

The structure of the particles in the individual preparation steps was documented by transmission electron microscopy (JEOL JEM-1011, voltage 80 kV) (FIG. 3). The observed changes correspond to the growth mechanism and document the presence of the expected structures, including the resulting “nanoshells”.

### Example 9

### In Situ Hybridization of Viral DNA in Tumour Cells

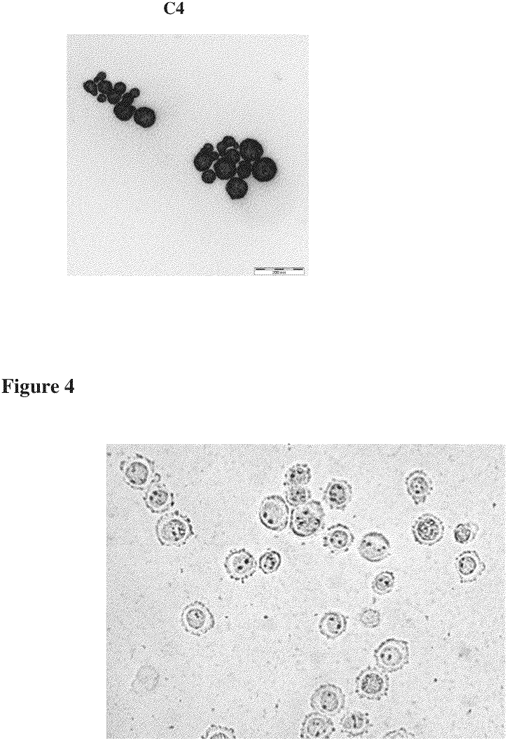

Neutravidinine-derivatized polymer-modified particles (A8) with high optical contrast in transmitted light were used to detect target DNA sequences in the tumour cells genome. In this case, they were tumour cells of cervical carcinoma (SiHa), which are characterized by the presence of 2-3 copies of the integrated form of the HPV16 virus per nucleus. The fixed cells or directly the tumor tissue were immobilized on a galss slide, the DNA was denatured and hybridized with a gene-specific DNA probe labeled with digoxigenin. Excess probe was washed away, and the cells were further incubated with rabbit anti-digoxigenin antibody followed by biotinylated anti-rabbit immunoglobulin antibody. The cells were then incubated with a solution of neutravidin-derivatized A8 particles that bind to biotin. The excess particles were washed away and the nuclei were stained with neutral red for better structural recognition of the cells. The specific HPV16 signal was visualized by light field microscopy as dark, high contrast spots on a light background located in the cell nucleus region (FIG. 4). This example demonstrates the use of modified particles for in vitro diagnostics.

### Example 10

### Use of Modified Particles for Detection of Phagocytic Activity

Measurement of the phagocytic activity of professionally phagocytic cells is an indicator of the parameters of non-specific cellular immunity. Its examination is routinely performed clinically from the patient’s blood cells by phagocytosis of fluorescently labeled mannosylated particles (e.g. latex, zymosan, etc.), staining microscopically, or on a flow cytometer. However, fluorescence flow cytometry is an expensive and sophisticated device that is not commonly available in every haematology laboratory. Due to the extreme scattering properties of the modified particles, they can be used to measure phagocytic activity, e.g. by flow cytometry in native, fluorescently unstained cells, by haemocytometry, or by light field microscopy with automated image analysis. The murine cell line J774A.1 (ATCC® TIB-67™) cultured in Dulbecco’s Modified Eagle’s Medium with 10% fetal bovine serum and antibiotics (100 U/100 µg penicillin /streptomycin) was used as model macrophages.

### Cytometric Detection

The day before the experiment, 6×10⁵ J774A.1 cells were seeded on a 60 mm culture dish. After 24 hours, fresh medium without particles (control), medium containing control modified particles B6 carrying only polymer (negative control) and modified particles B7 with bound mannose (both particles at a final concentration of 7.3 pmol.1⁻¹) were added. Cells were harvested at 0, 1, 3 and 6 h intervals after the addition of medium with or without particles and subsequently measured without fixation by light scattering detection. The results are summarized in FIG. 5. It can be seen that the mannosylated particles B7 were selectively and intensively taken up by macrophages, the uptake was time-dependent, and that the cytometric method without the use of fluorescent labeling was very sensitive for the detection of phagocytic activity.

### Detection by Light Field Microscopy

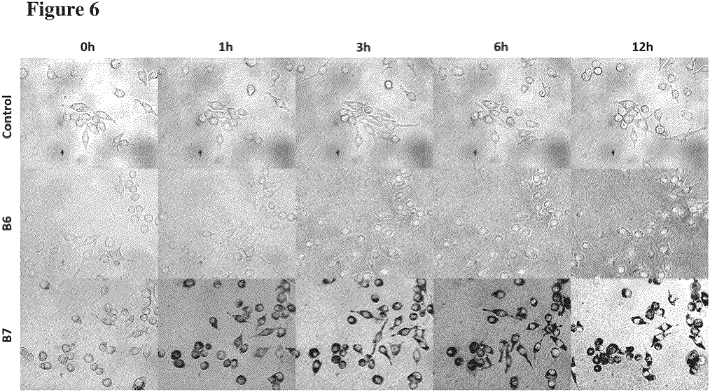

The day before the experiment, 1×10⁴ J774A.1 cells were seeded in a 96-well plate. After 24 hours, fresh medium without particles (control), medium containing control modified particles B6 carrying only polymer (negative control) and modified particles B7 with bound mannose (both particles at a final concentration of 7.3 pmol.1⁻¹) were added. Live cell imaging at 0, 1, 3, 6, 12, and 18 h after addition of medium with or without particles was performed using a high-capacity analysis in a microscope discovery system Cell Voyager CV7000 (Yokogawa, Japan) at 37° C. in 5% CO₂ in light field mode. The results are summarized in FIG. 6. As in the case of cytometric detection, the microscopic method without the use of fluorescent labeling was very sensitive for the detection of phagocytic activity. Mannosylated particles B7 were selectively and intensively taken up by macrophages and uptake was time-dependent.

### Example 11

### Detection of CD3+ and CD4+ T Lymphocytes

This Example demonstrates the use of particles C9 and C10 modified with monoclonal antibodies CD3 or CD4, respectively, as a staining tool for CD3+ or CD4+ T lymphocytes, respectively. The number of CD4+ T lymphocytes in blood is supressed in the patients who suffer form Human Immunodeficiency Virus (HIV). Counting CD4+ T lymphocytes is one of the most useful parameters for following the progress of HIV disease.

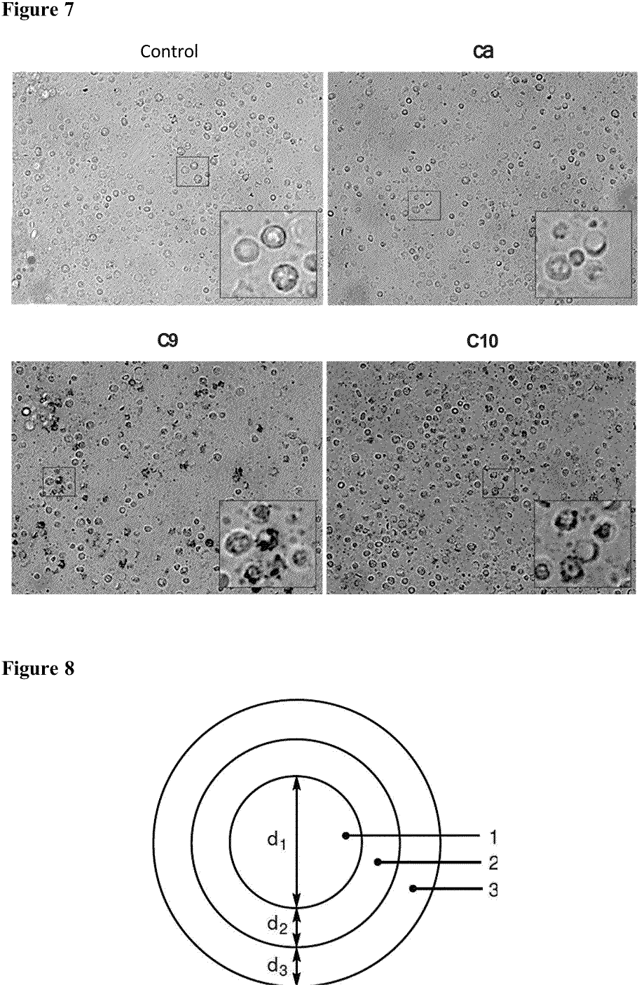

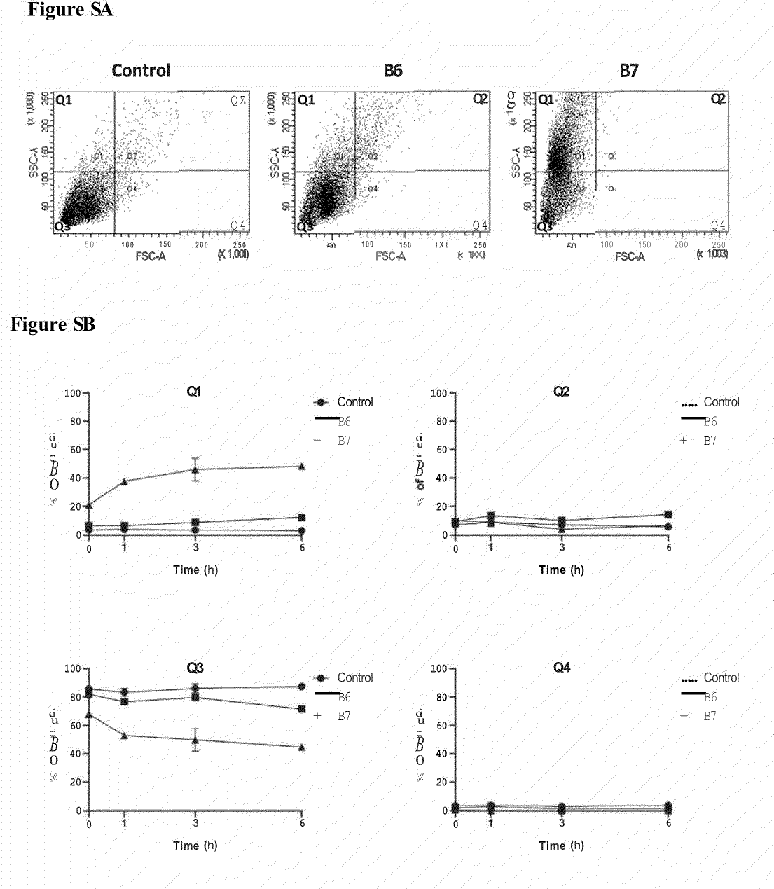

PBMC cells were isolated from whole blood using density gradient centrifugation (Ficoll™), seeded in CellCarrier-96 well plate (PerkinElmer) at 5 × 10⁴ cells/well and incubated with C9 particles modified with CD3 monoclonal antibody or with C10 particles modified with CD4 monoclonal antibody for 48 h at 4° C. The cells were imaged using PerkinElmer Operetta imaging system. Bright field images were captured by 40x long WD objective. All images were post-processed using integrated Operetta system Harmony 4.1 and ImageJ software. The results are summarized in FIG. 7. The microscopic method without the use of fluorescent labeling was very sensitive for the detection of the specific cells displaying the target receptors. The control particles C8, which did not contain any antibody, did not stained the cells. In contrast, the fraction of cells containing CD3 receptor was stained by particles C9, and the fraction of cells containing CD4 receptor was stained by particles C10 (FIG. 7). After further optimisation and validation this method can be used as a quantitative tool for counting the number of circulating the level in whole blood.

### INDUSTRIAL APPLICABILITY

The present invention solves the problem of direct detection in in vitro diagnostics by light extinction using surface-modified particles with a non-metallic core coated with a metal layer on which a biocompatible polymer brush is attached. Their application allows the direct detection of biomolecules including nucleic acids, proteins, polysaccharides and glycoproteins in biological samples. By biological sample is meant, for example, blood, blood plasma, blood serum, urine, semen, tears, saliva, mucus, stool, sweat, swab, lymph, cerebrospinal fluid, cell suspension or tissue sample.

1. Surface-modified particles, characterized in that they comprise a core, an inner shell and an outer shell, wherein
the core is formed of silica or the core is hollow; and the core has a diameter d₁ in the range of 20 nm to 1 µm,
the inner shell consists of a layer of metal M, said layer having a thickness d₂ in the range of 2 to 60 nm,
the outer shell has a thickness d₃ in the range of 2 to 200 nm, and the outer shell consists of a layer of a polymer of the general formula 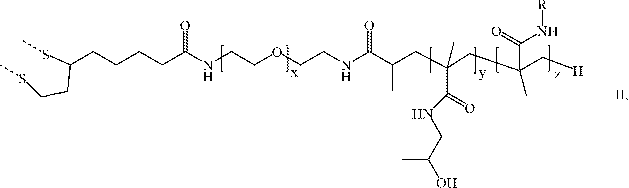
wherein x = 2 to 50, y = 5 to 5000, z = 0 to 2000, z/y = 0 to 0.4;
R are the same or different on each occurence, wherein each R is independently selected from the group consisting of —(CH₂)ₙ—C≡CH, —(CH₂)ₘ—N₃, —(CH₂)ₙ—NH₂, —(CH₂)ₙ—COOH, 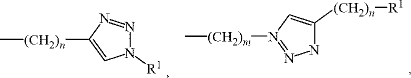 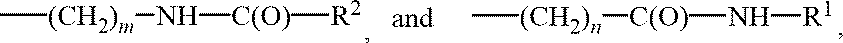
wherein n = 1 to 4, m = 2 to 5;
R¹ is selected from the group consisting of 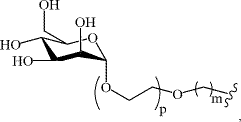 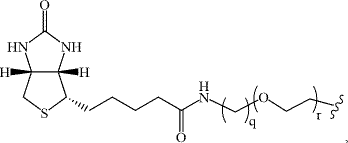
and a fluorophore,
wherein p = 0 to 24, q = 2 or 3, r = 0 to 24;
R² is selected from the group consisting of 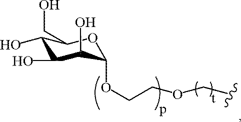 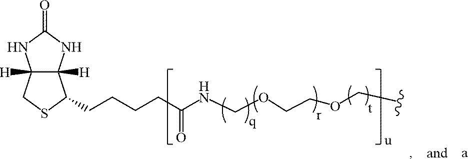
fluorophore,
wherein p =0 to 24, q = 2 or 3, r= 0 to 24, t = 1 to 4, u = 0 or 1;
wherein when R¹ is 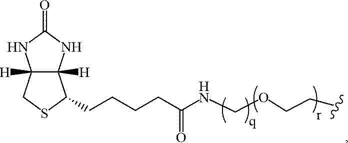
the biotin moiety is optionally conjugated to a biotin-binding protein such as neutravidin, streptavidin, or avidin, via formation of a non-covalent attachment between biotin and the biotin-binding protein;
and wherein when R¹ is 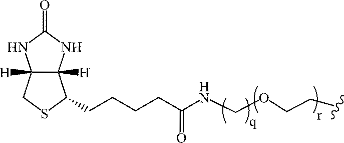
and it is conjugated to the biotin-binding protein, a biotin-modified biomolecule is optionally non-covalently attached to the biotin-binding protein via formation of a non-covalent attachment between the biotin-binding protein and the biotin moiety of the biotin-modified biomolecule;
wherein the polymer of the general formula II is attached to the surface of the inner shell by means of its sulphur atoms forming an M-S bond with the metal atoms of the inner shell, as depicted by the dashed bonds in the formula II.

2. Surface-modified particles according to claim 1, characterized in that the inner shell has a thickness d₂ in the range of 3 to 25 nm, more preferably 5 to 20 nm; and/or the outer shell has a thickness d₃ in the range of 5 to 100 nm, more preferably 10 to 60 nm.

3. A process for the preparation of surface-modified particles according to claim 1, wherein particles comprising a core and an inner shell are reacted with a compound of formula III 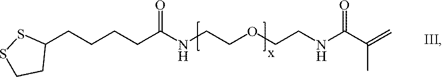
wherein x is as defined in claim 1,
wherein the compound of formula III is optionally in a mixture with lipoic acid in a molar ratio of lipoic acid: compound of formula III = 2:1 to 6:1, preferably 4:1,
and the product is subsequently contacted with monomer of formula IV under free radical polymerization conditions 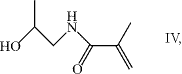
wherein the monomer of formula IV optionally contains an admixture of 0 to 40 mol% of monomer of formula V 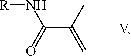
wherein R is as defined in claim 1, to form a polymer of formula II 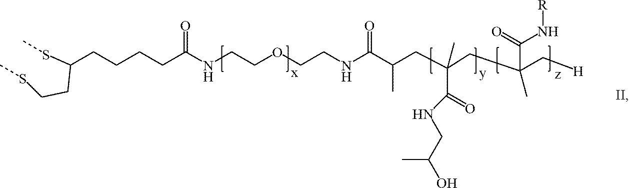
wherein R, x, y, z are as defined in claim 1,
attached to the inner shell, thereby forming the outer shell.

4. The process for the preparation of surface-modified particles according to claim 1, wherein a compound of formula IIA 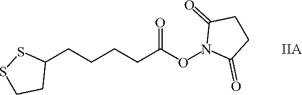 [SMILES: O=C(CCCCC1CCSS1)ON1C(=O)CCC1=O]
is reacted with polymer of formula IIB 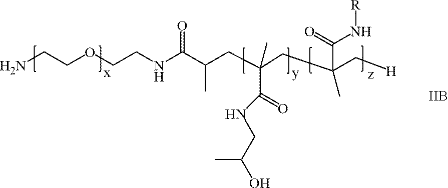
to form polymer of formula IIC 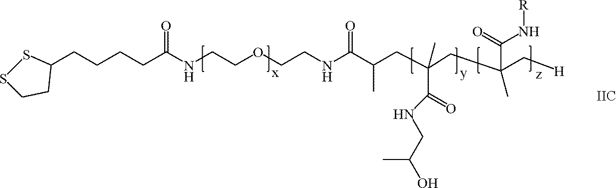
wherein R, x, y, z are as defined in claim 1,
and the polymer of formula IIC is subsequently reacted in an aqueous medium, optionally in a mixture with lipoic acid in a molar ratio of lipoic acid : polymer of formula IIC = 2:1 to 6:1, preferably 4:1, with particles comprising a core and an inner shell as defined in claim 1,
to form particles with polymer II 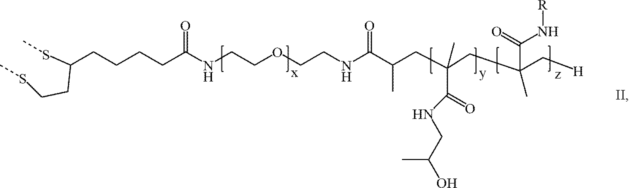
bound to the inner shell and forming the outer shell.

5. A process according to claim 3, wherein the particles comprising the core and the inner shell contain gold inner shell and are prepared by the following steps:
in a first step, silica cores are modified by reaction with trialkoxysilane derivatives of formula R⁴Si(R³)₃, wherein R⁴ is selected from C₂-C₄ alkyl terminally substituted with mercapto or amino group, and R³ are selected from the group consisting of —OCH₃ and —OCH₂CH₃,
in a second step, gold nanoparticles with a diameter of less than 5 nm are bound to the thus modified core particles,
in a third step, the resulting particles, formed by a core with bound gold nanoparticles, are reacted with [AuCl₄⁻] in the presence of a reducing agent, thus forming an inner shell.

6. The process according to claim 5, wherein in the first step the trialkoxysilane derivatives are selected from the group consisting of (3-mercaptopropyl) trimethoxysilane, (3-mercaptopropyl)triethoxysilane, (3-aminopropyl)trimethoxysilane, and (3-aminopropyl)triethoxysilane.

7. The process according to claim 5, wherein in the third step, the reducing agent is selected from the group consisting of carbon monoxide, hydroxylamine, hydrazine, methylhydrazine, ascorbic acid, formaldehyde and acetaldehyde.

8. The process according to claim 3, wherein the particles comprising the core and the inner shell contain gold inner shell and are prepared by the following steps:
in a first step, (3-aminopropyl)triethoxysilane or (3-aminopropyl)trimethoxysilane is stirred with water,
in a second step, HAuCl₄ is added followed by addition of NaBH₄, to form particles, preferably HAuCl₄ and NaBH₄ are added in the form of solution(s),
in a third step, the formed particles are stabilized by bovine serum albumine.

9. The process according to claim 1 for in vitro detection of biomolecules in biological samples, wherein the detection includes interaction of modified particles with said biomolecules, wherein the biomolecules are selected from the group consisting of nucleic acids, proteins, polysaccharides and glycoproteins.
